(19) United States
(12) Patent Application Publication
(10) Pub. No.: US 2004/0042985 A1
(43) Pub. Date: Mar. 4, 2004

(54) Dry fluid compositions and products thereof

(76) Inventor: Peter Boncelet, Ormond Beach, FL (US)

Correspondence Address: John P. Blasko, Fox Rothschild O'Brien & Frankel, LLP, P.O. Box 5231, Princeton, NJ, 08543-5231, US

(21) Appl. No.: 10/233,833

(22) Filed: Aug. 30, 2002

(51) Int. Cl. A61K 7/32

(52) U.S. Cl. 424/65

(57) ABSTRACT

Dry fluid compositions for use in cosmetic and personal hygiene products are disclosed. The dry fluid compositions are comprised of a base oil and a combination of at least two esters compounds selected from lauryl laurate, cetyl palmitate, stearyl behenate, and cetyl laurate. Inexpensive oils and petrolatum are used in place of more expensive silicones but the dry fluid compositions do not sacrifice the benefits of a silicone oil. The dry fluid compositions are most often used as part of an emulsion in personal hygiene products such as antiperspirants and deodorants or by itself as a body oil product.

### FIELD OF THE INVENTION

This invention relates to dry fluid compositions comprised of a mineral, animal, vegetable, white or petrolatum base oil combined with two or more high melting point aliphatic acid ester compounds. The dry fluid compositions can be used alone as a massage oil, bath oil or the like, or in combination with, for example, antiperspirant actives in personal hygiene products such as antiperspirants and deodorants.

### BACKGROUND OF THE INVENTION

Antiperspirant and deodorant products, which include antiperspirant or deodorant actives, and massage and bath oils are widely used everyday. These products usually include non-volatile silicone oils or silicone derivatives which are expensive to produce and have a “heavy” feeling to the touch.

A large variety of commercially available antiperspirant and deodorant formulations have been described in the patent literature. The antiperspirant “stick” products typically fall within three categories, namely suspension sticks, emulsion sticks and solution sticks. Patents and patent applications which have discussed the use and standard preparation of these products include: U.S. Pat. No. 6,352,688, U.S. Pat. No. 6,387,357, U.S. Pat. No. 4,673,570, U.S. Pat. No. 5,008,103, U.S. Pat. No. 5,401,870, U.S. Pat. No. 5,292,503, U.S. Pat. No. 4,980,156, U.S. Pat. No. 4,673,570, U.S. Patent Application No. 20020039563 and U.S. Patent Application No. 20020034481 all of which are incorporated herein by reference. Also various physical forms may be used such as solids (for example, wax and sticks), semi-solids (for example, gels and creams), liquids (for example, roll-on products) and sprays (both aerosol and non-aerosol).

Conventionally, emulsion-type sticks have an external or oil phase substantially of silicone material and an internal or polar phase which contains the active ingredient. These emulsions may be used to form clear gel, soft solid or roll-on products. In solution sticks, the antiperspirant is typically dissolved in a liquid carrier phase comprising water or a water-miscible organic solvent which is structured with naturally-occurring or synthetic waxy materials. In suspension sticks, a particulate antiperspirant active material is suspended in a structured carrier.

In recent years a strong emphasis has been placed on improving both the performance and the aesthetics of these products. One of the particular problems is trying to obtain an emulsion which is easier and less expensive to make. Previous attempts have not successfully overcome the problems of improving efficacy and achieving satisfactory formation of emulsions.

Thus, it is an object of this invention to provide improved emulsions containing an external phase which exhibit improved efficacy and, at the same time, have a stability profile that allows for satisfactory stability on the shelf.

It is another object of the present invention to provide a dry fluid with enhanced aesthetics such as smoothness in application, low tack and dry feel.

It is also an object of this invention to provide a dry fluid to be used in an emulsion which is easier and less expensive to manufacture.

### SUMMARY OF THE INVENTION

In one aspect, the present invention comprises dry fluid compositions having a base oil of mineral oil, vegetable oil, animal oil, white oil or petrolatum combined with one or more high melting point aliphatic acid ester compounds. The invention uses these inexpensive oils and petrolatum in place of more expensive silicones oils and silicone wax, but does not sacrifice the benefits of a silicone. Most advantageously, the dry fluids have a silicone oil texture without the use of a silicone.

The compositions of the present invention have a plurality of uses including silicone replacements, silicone extenders, feel modifiers and aesthetic feel improvers. Basically, it has been found that where a formulation would use a silicone oil, the dry fluid compositions of the present invention may be used to replace all or part of the silicone oil component. The use of these dry fluids completely removes the negative feel characteristics of the base i.e., heavy, oily, greasy, etc., without sacrificing the benefits of improved moisture, barrier, lubrication, and emolliency.

The dry fluid compositions can also be used in formulations of baby oils, massage oils, sunscreen oils, lipsticks, makeup sticks, antiperspirant sticks, antiperspirant gels, antiperspirant roll-on's, antiperspirant soft solids, deodorant sticks, deodorant gels, deodorant roll-ons, deodorant soft solids, cosmetic lotions, sunscreen lotions, and the like.

It has been found that the dry fluids of the present invention are particularly well suited for use in the antiperspirant stick and deodorant stick formulations. Thus, in another aspect, the present invention comprises antiperspirant and deodorant formulations.

### DETAILED DESCRIPTION OF THE INVENTION

The invention comprises dry fluids having a base oil of either mineral oil, vegetable oil, animal oil, white oil or petrolatum combined with two or more high melting point aliphatic acid ester compounds.

Non-limiting examples of oils suitable for use as a base oil are petrolatum, mineral oil, white oil, animal oils—emu, cod liver, fish oil, marine, menhaden, shark, squalane, and the like—and vegetable oils—almond, sweet almond, apricot kernel, avocado, borage, brazil nut, camelina, canola, carrot, castor, coconut, corn, cottonseed, evening primrose, flaxseed, grapeseed, hazelnut, hemp, jojoba, kukui nut, kiwi, macadamia nut, oat, olive, palm, parsley seed, peanut, pecan, pistachio, pumpkinseed rice bran, rose hip seed, rosemary, safflower, sesame, soybean, sunflower, walnut, wheat germ, and the like—and combinations thereof.

The preferred high melting point aliphatic acid ester compounds are lauryl laurate, cetyl palmitate, stearyl behenate, and cetyl laurate and combinations thereof.

Results from experiments have illustrated the combination of lauryl laurate and cetyl palmitate is particularly effective. It is believed that this combination of ester compounds is most effective based on their molecular weight and melting points which permit better solubility and dispersion of the esters into the base oil resulting in a better texture for the emulsion.

The dry fluid compositions are comprised of a combination of ester compounds in an amount of 0.1-15% by total weight of the composition, more preferably, in an amount of 2-8% by weight. The ratio by weight of a first ester compound to a second ester compound in the combination is preferably in the range of 1:20 to 20:1, more preferably 1:10 to 10:1 and most preferably 2:3 to 3:2.

The process for manufacturing the dry fluid compositions is to heat the ester compounds and blend them with the base oil. Specifically, the various ester compounds are heated to a liquid phase, percentages weighed, and thereafter added and mixed with the base oil.

Advantageous uses of the dry fluids are described below, but in no way should these examples be construed as limiting. COSMETIC AND PERSONAL HYGIENE PRODUCTS The dry fluid compositions may be substituted for non-volatile silicon oils in a wide variety of cosmetic and personal hygiene products. They are useful in antiperspirant products and may be emulsified with an “active” ingredient to form a stick-type product.

If the product is an antiperspirant, it will contain an antiperspirant active.

Antiperspirant actives, are preferably incorporated in an amount of from 0.5-60%, particularly from 5 to 30% or 40% and more preferably from 5 or 10% to 30 or 35% of the weight of the composition of the final product.

Antiperspirant actives for use herein are often selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Various antiperspirant active materials can be utilized according to the present invention provided they are soluble at a suitable concentration in the internal phase. These include conventional aluminum and aluminum/zirconium salts, as well as aluminum/zirconium salts complexed with a neutral amino acid such as glycine, as known in the art. A variety of active ingredients are disclosed in U.S. Pat. No. 6,387,357 and U.S. Patent Application No. 20020039563, the contents of each of which are incorporated herein by reference in their entirety. Deodorant active materials can also be included such as fragrances, anti-microbial agents and masking agents.

The dry fluid composition may constitute 0.1 to 70% (preferably 15 to 45%) of the antiperspirant emulsion. Optionally, non-volatile silicone material (particularly 0.1 to 45%, more particularly 0.1 to 20%) and volatile silicone material (particularly 0.1 to 60%, more particularly 0.1 to 45%) may be used in combination with the dry fluid composition.

### EXAMPLES

The following examples are offered as illustrative of the invention and are not to be construed as limitations thereof.

Compositions using the present invention may be made by the techniques described below and in the incorporated references. Weights by percentage are shown in Table 1. In general, the external and internal phases are formed separately using heating with the addition of a non-ionic emulsifier as needed.

Preparation of the External Phase

The ingredients to be used in the external phase are heated to a liquid consistency, weighed and combined in a suitable vessel. The mixture is stirred at about 500 rpm for 15-20 minutes using an overhead mixer. In the preferred embodiment, lauryl laurate which has a melting point of 24° C. is heated to approximately 30° C. and cetyl palmitate which has melting point of 48° C. is heated to approximately 55° C. so each will be in a liquid phase. These esters are combined with a mineral oil, a vegetable oil or petrolatum, heated to a liquid phase, and thereafter, mixed (as discussed above).

Preparation of the Internal Phase

The ingredients of the internal phase are mixed for a time sufficient to achieve homogeneity. The antiperspirant active used (for example, Westchlor Zr 35 BX3 (for example, 43% aluminum-zirconium glycinate in water) is weighed into a large beaker equipped with an overhead stirrer. Other internal phase ingredients are then added while stirring.

The fragrance (if used) is added last and may be added to the external phase (although it may be added to either the external phase or the internal phase if alcohol is used in the formulation) or the final formula prior to homogenization. Various procedures for preparation of the internal phase are well known in the art and have been incorporated herein by reference.

Preparation of the Emulsion

The internal phase is added to the external phase under procedures known in the art which have been incorporated herein by reference. It is believed that the more homogeneous the composition is and the more uniform the particle size, the better properties of the composition.

TABLE 1

| Ingredient | Ex. 1 | Ex. 2 | Ex. 3 | Ex. 4 | Ex. 5 |
|---|---|---|---|---|---|
| EXTERNAL | | | | | |
| PHASE | | | | | |
| Cetyl | 1.00 | 1.25 | 1.00 | 1.25 | 1.75 |
| Palmitate | | | | | |
| Lauryl Laurate | 1.50 | 1.25 | 1.50 | 1.25 | .75 |
| Base | 22.5 | 22.5 | 10 | 10 | 22.5 |
| Silicone | | | 12.5 | 12.5 | |
| INTERNAL | | | | | |
| PHASE | | | | | |
| Antiperspirant | 35 | 40 | 50 | 60 | 55 |
| Active | | | | | |
| Water | 40 | 35 | 25 | 15 | 10 |
| Fragrance | | | | | 10 |

Examples of formulations of sticks that can be made with compositions of the present invention include combining the appropriate ingredients by conventional techniques. The percents given below are weight percents based on the total weight of the composition. Antiperspirant Stick “A”

External Phase

1) 0.5-8.0% (preferably 2-6%) of a siloxane polyamide gelling agent such as those described in patent application U.S. Ser. No. 08/904,709, filed Aug. 1, 1997, and incorporated herein by reference in its entirety;

2) 0-60% (preferably 20-40%) of a silicone fluid selected from the group consisting of D4, D5 and D6 cyclomethicones and mixtures thereof where the D# represents the number of siloxane units in the ring;

3) 15-60% (preferably 20-40%) of a dry fluid composition according to the present invention

4) 0-20% (preferably 7-15%) of at least one cosmetic ingredient selected from the group consisting of non-volatile emollients, for example, C8-C22 fatty alcohols, C12-C36 fatty esters, C8-C18 alkyl benzoates, and linear polysiloxanes;

5) 0-10% (preferably 3-7%) of a cosmetically acceptable surfactant selected from the group consisting of cationic surfactants (for example, cetyltrimethyl ammonium chloride), nonionic surfactants (for example, polysorbate 20), anionic surfactants (for example, sodium lauryl sulfate), amphoteric surfactants (for example, cocamidopropyl hydroxysultaine), dimethicone copolyols and polyether ethoxylates;

6) 0-3% (preferably 1-2%) of a fragrance; Internal Phase

7) 5-40% (preferably 15-25%) of water and/or water miscible solvents;

8) 0-20% (preferably 3-15%) of an amino acid (preferably glycine) or mixtures of amino acids;

9) 10-25% (preferably 15-25%) of an antiperspirant active;

10) 0-5% (preferably 1-2%) of a cosmetically acceptable surfactant selected from the group consisting of cationic surfactants (for example, cetyltrimethyl ammonium chloride), nonionic surfactants (for example, polysorbate 20), anionic surfactants (for example, sodium lauryl sulfate), amphoteric surfactants (for example, cocamidopropyl hydroxysultaine), dimethicone copolyols and polyether ethoxylates.

Antiperspirant Stick “B”

1) 25-70% (particularly with a high oil content of 25-50%, and more particularly 30-45%) of an external phase comprising:

(i) 0-10% (on an actives basis) of at least one elastomer which is a cyclomethicone (and) dimethicone crosspolymer, preferably with a nonvolatiles content of 8-18% (particularly 10-14% and most particularly 12-13%) in cyclomethicone (for example a D4 or D5 cyclomethicone), (an example of such a crosspolymer composition being DC-9040 from Dow Corning Corporation (Midland, Mich.) with other types of such crosspolymers (also called elastomers) being described in U.S. Pat. No. 5,654,362, incorporated by reference herein as to the description of such polymers and methods of making such polymers);

(ii) 0-5% (particularly 0.1-1.0%) of a silicone copolyol having an HLB value (hydrophilic lipophilic balance)≦8;

(iii) 0.1-68% of a volatile silicone selected in an amount to complete the external phase;

(iv) 0-10% (particularly 0-5%) of a cosurfactant or emulsifier having an HLB value in the range of 1-15;

(v) 0-5% of a non-volatile silicone;

(vi) 0.1-70% (particularly 15-45%) of the dry fluid composition and

2) 30-75% (particularly 50-75%) of an internal phase comprising:

(i) 7-25% (on an anhydrous actives basis excluding the waters of hydration) of an antiperspirant active preferably added as a solution of active in water and/or water+glycol mixture as a solvent;

(ii) 0-10% ethanol;

(iii) additional water to complete the internal phase;

(iv) 0-5% of an antimicrobial agent; and

(v) 0-5% of an ionizable salt.

Examples of formulations of gels that can be made with compositions of the present invention include a transparent antiperspirant gel made by combining:

Gel

External Phase

1) 0-20% (preferably 4-12%) cyclomethicone;

2) 0-2% (preferably 0.8-1.5%) dimethicone copolyol;

3) 0-20% (preferably 7-12%) linear silicones (for example, dimethicone);

4) 5-30% (preferably 9-20%) dry fluid composition

Internal Phase

5) 25-60% (preferably 30-45%) water;

6) 5-40% (preferably 7-30%) water miscible solvents;

7) 0-20% (preferably 3-15%) of an amino acid (preferably glycine) or mixtures of amino acids;

8) 10-25% (preferably 15-23%) of an antiperspirant active;

9) 0-2% (preferably 0.5-1%) of a cosmetically acceptable surfactant selected from the group consisting of cationic surfactants (for example, cetyltrimethyl ammonium chloride), nonionic surfactants (for example, polysorbate 20), anionic surfactants (for example, sodium lauryl sulfate), amphoteric surfactants (for example, cocamidopropyl hydroxysultaine), dimethicone copolyols and polyether ethoxylates.

Examples of formulations of creams that can be made with compositions of the present invention include creams made by combining:

Cream

External phase

1) 0-10% (preferably 2-5%) Cyclomethicone;

2) 0.1-3% (preferably 0.4-1.0%) of triglycerides such as mono-, di-, or triglycerides' and mixtures thereof (for example, glycerol monostearate);

3) 4-15% (preferably 8-12%) cosmetically acceptable surfactants/emulsifiers (for example a member selected from the group consisting of cationic surfactants (for example, cetyltrimethyl ammonium chloride), nonionic surfactants (for example, polysorbate 20), anionic surfactants (for example, sodium lauryl sulfate), amphoteric surfactants (for example, cocamidopropyl hydroxysultaine), dimethicone copolyols and polyether ethoxylates;

4) 3-8% (preferably 3.5-6.5%) of a C8-C22 fatty alcohol;

5) 1-8% (preferably 3-6%) of a dry fluid composition

Internal Phase

6) 40-85% (preferably 50-70%) water;

7) 0-20% (preferably 3-15%) amino acid (preferably glycine);

8) 10-25 (preferably 15-25%) of an antiperspirant active.

Examples of sprays that can be made with the compositions of the present invention include a water-based pump spray made by combining:

Spray

1) 35-85% (preferably 50%-75%) water;

2) 3-7% (preferably 4-5%) water soluble emollient;

3) 0-3% (preferably 1-2%) of a cosmetically acceptable surfactant selected from the group consisting of cationic surfactants (for example, cetyltrimethyl ammonium chloride), nonionic surfactants (for example, polysorbate 20), anionic surfactants (for example, sodium lauryl sulfate), amphoteric surfactants (for example, cocamidopropyl hydroxysultaine), dimethicone copolyols and polyether ethoxylates;

4) 10-25% (preferably 15-25%) of an antiperspirant active;

5) 0-20% (preferably 3-15%) of an amino acid (preferably glycine) or mixtures of amino acids;

6) 3-10% (preferably 4-8%) dry fluid composition.

Examples of a water based roll-on that may be made with the compositions of the present invention include a composition made by combining:

Roll-on

1) 30-85% (preferably 45-70%) water;

2) 0.5-3% (preferably 1-2%) of magnesium aluminum silicate;

3) 0.5-10% (preferably 3-7%) of a cosmetically acceptable surfactant selected from the group consisting of cationic surfactants (for example, cetyltrimethyl ammonium chloride), nonionic surfactants (for example, polysorbate 20), anionic surfactants (for example, sodium lauryl sulfate), amphoteric surfactants (for example, cocamidopropyl hydroxysultaine), dimethicone copolyols and polyether ethoxylates;

4) 0-5% (preferably 1-2%) of water miscible solvent(s);

5) 10-25% (preferably 15-25%) of an antiperspirant active;

6) 0-20% (preferably 3-15%) of an amino acid (preferably glycine) or mixtures of amino acids;

7) 3-10% (preferably 4-8%) dry fluid composition.

Examples of water-in-silicone antiperspirant roll-ons are those made by combining:

External Phase

1) 0-50% (preferably 15-25%) cyclomethicone;

2) 0-2% (preferably 0.8-1.5%) dimethicone copolyol;

3) 10-50% (preferably 20-35%) dry fluid composition

Internal Phase

4) 30-50% (preferably 38-45%) water;

5) 5-40% (preferably 15-25%) water miscible solvents;

6) 0-20% (preferably 3-15%) of an amino acid (preferably glycine) or mixtures of amino acids;

7) 10-25 (preferably 15-25%) of an antiperspirant active;

8) 0-2% (preferably 0.5-1%) of a cosmetically acceptable surfactant selected from the group consisting of cationic surfactants (for example, cetyltrimethyl ammonium chloride), nonionic surfactants (for example, polysorbate 20), anionic surfactants (for example, sodium lauryl sulfate), amphoteric surfactants (for example, cocamidopropyl hydroxysultaine), dimethicone copolyols and polyether ethoxylates.

Massage Oils

The dry fluids of the present invention can be used alone as a massage oil or a “baby oil”.

### EXAMPLES

Table 2 illustrates the composition of the dry fluids when used as the product alone in percentage by weight. As the use of the dry fluid would not be a silicone substitute, it could be 100% of the product. Various amounts of silicone can be added if desired. Preparation of the dry fluid is in the same manner as when used as an external phase of a deodorant or antiperspirant product. The ratio of ester compounds to base is also substantially equivalent as used in an antiperspirant formulation.

TABLE 2

| Ingredient | Ex. 6 | Ex. 7 | Ex. 8 |
|---|---|---|---|
| Cetyl | 5.0 | 6.0 | 5.0 |
| Palmitate | | | |
| Lauryl Laurate | 5.0 | 4.0 | 5.0 |
| Base | 90.0 | 90.0 | 80.0 |
| Silicone | | | 10.0 |

What is claimed is:

1. A dry fluid composition comprising:
(a) 0.1 to 15 wt % of a combination of at least two high molecular weight aliphatic esters selected from the group consisting of lauryl laurate, cetyl palmitate, stearyl behenate, and cetyl laurate; and
(b) 85 to 99.9 wt % of a base oil selected from the group consisting of mineral oils, vegetable oils, petrolatum and combinations thereof.

2. The dry fluid composition of claim 1, wherein said at least two esters are lauryl laurate and cetyl palmitate.

3. The dry fluid composition of claim 2, wherein the ratio by weight of lauryl laurate to cetyl palmitate is in the range of 1:20 to 20:1.

4. The dry fluid of claim 1, wherein said base is selected from the group consisting of mineral oil, vegetable oils, animal oils, white oil, petrolatum and mixtures thereof.

5. A antiperspirant and/or deodorant composition in the form of an emulsion comprising:
(a) an external phase comprising:
(i) the dry fluid composition of claim 1; and
(b) an internal phase comprising an antiperspirant active.

6. An antiperspirant and/or deodorant composition in the form of an emulsion comprising:
(a) 25-70% of an external phase component comprising:
(i) 0.1-70% of a dry fluid component comprising by percent weight:
(a) 0.1-15% of a combination of at least two high molecular weight aliphatic esters selected from the group consisting of: lauryl laurate, cetyl palmitate, stearyl behenate, and cetyl laurate; and
(b) 85-99.9% oil selected from the group consisting of petrolatum, mineral oil, white oil, animal oils, and vegetable oils; and
(b) 30-75% of a internal phase component comprising:
(i) 7-25% of an antiperspirant active;
(ii) 0-10% ethanol;
(iii) additional water to complete internal phase;
(iv) 0-5% of an antimicrobial agent; and
(v) 0-5% of an ionizable salt.

7. The composition of claim 6, further comprising in the external phase component:
(ii) 0.1-45% of a non-volatile silicone
(iii) 0.1-60% of a volatile silicone material
(iv) 0-10% of a cosmetically acceptable surfactant selected from the group consisting of cationic surfactants, nonionic surfactants, anionic surfactants, amphoteric surfactants, dimethicone copolyols and polyether ethoxylates.
